(19) United States
(12) Patent Application Publication
(10) Pub. No.: US 2013/0059135 A1
(43) Pub. Date: Mar. 7, 2013

(54) PRINTING COMPOSITION AND A PRINTING METHOD USING THE SAME

(71) Applicants: Sang Ki Chun, Daejeon (KR); In-Seok Hwang, Daejeon (KR); Dong Wook Lee, Daejeon (KR); Yong Koo Son, Daejeon (KR)

(72) Inventors: Sang Ki Chun, Daejeon (KR); In-Seok Hwang, Daejeon (KR); Dong Wook Lee, Daejeon (KR); Yong Koo Son, Daejeon (KR)

(73) Assignee: LG CHEM LTD, SEOUL (KR)

(21) Appl. No.: 13/698,617

(22) PCT Filed: Jun. 3, 2011
(86) PCT No.: PCT/KR11/04077

§ 371 Date: Nov. 16, 2012

(30) Foreign Application Priority Data
Aug. 16, 2010 (KR) 10-2010-0078973

(51) Int. Cl. C09D 5/00; C07D 207/06; H01B 1/12; C09D 133/08; C09D 141/00; C09D 163/00; C09D 175/04; C09D 167/00; C09D 171/00; C09D 171/10; C09D 183/00; C09D 179/08; B41M 1/00; B32B 3/10; C07D 233/58

(52) U.S. Cl. 428/206; 548/335.1; 548/579; 106/287.2; 106/287.21; 252/500; 524/560; 524/547; 523/400; 524/590; 524/599; 524/611; 524/588; 524/606; 101/483; 428/195.1

(57) ABSTRACT

The present invention relates to a printing composition including an ionic liquid, a printing method using the same, and a pattern formed by using the same. The printing composition according to the exemplary embodiment of the present invention is useful in providing a pattern of a fine line width.

### DISCLOSURE

1. Technical Field

The present invention relates to a printing composition and a printing method using the same. More particularly, the present invention relates to a printing composition that can implement a fine line width and a printing method using the same. This application claims priority from Korean Patent Application No. 10-2010-0078973 filed on Aug. 16, 2010 in the KIPO, the disclosure of which is incorporated herein by reference in its entirety.

2. Background Art

As a method for forming a pattern, there are an indirect pattern forming method such as a photolithography method and a direct pattern forming method such as a method in which a pattern is directly printed on a target substrate.

The indirect pattern forming method may be performed by the following process. First, a photoresist pattern is formed by uniformly coating photoresist on a film formed on a substrate and selectively exposing and developing the photoresist. Subsequently, the pattern is transferred by etching the above film by using a photoresist pattern as a mask. Thereafter, the photoresist is removed by a stripping solution.

The indirect pattern forming method does not use a photoresist material and a stripping solution in addition to a film on which a pattern will be formed, thereby increasing a process cost due to a cost of the photoresist material and the stripping solution and a cost of removing the photoresist material and the stripping solution. In addition, there is a problem in that an environment is polluted by removing the above materials. In addition, since the indirect method has the large number of processes and is complicated, much time and many costs are required, and in the case where the photoresist material is not sufficiently stripped, there is a problem in that defects occur in the final products.

Meanwhile, in the case of the method for directly printing the pattern, whether or not the printing property according to drying of the printing composition exists during the printing process largely affects implementation of a fine line width. In detail, in the case of directly printing the pattern of the fine line width, since a line height and a line width become small as compared to the case where the pattern having a thick line width is printed, the solvent is volatilized into the air or is absorbed into a silicon blanket during the printing process, thus maximizing a problem of drying the printing composition. In the case where the drying of the solvent into the air is too fast, as the ink composition is dried on cliché in doctoring, a residual film is generated, such that in pattern transferring, for example, in an off, a problem of printing non-uniformity occurs. Meanwhile, if the absorption of the solvent into the silicon blanket is too much, poor transferring into the substrate may occur in setting because of the drying of the printing composition on the surface of the blanket.

### DISCLOSURE

### Technical Problem

The present invention has been made in an effort to provide a printing composition providing a pattern having a fine line width and a printing method using the same.

### Technical Solution

An exemplary embodiment of the present invention provides a printing composition comprising an ionic liquid.

Another exemplary embodiment of the present invention provides a printing method using the printing composition.

Yet another exemplary embodiment of the present invention provides a pattern formed by using the printing composition.

### Advantageous Effects

According to exemplary embodiments of the present invention, a printing composition, unlike a known ink composition, improves a drying problem according to volatilization of a solvent into the air and an absorption problem into a blanket, thereby being usefully used in formation of a pattern of a fine line width.

### BRIEF DESCRIPTION OF DRAWINGS

FIG. 1 is a picture that illustrates a printing result according to Comparative Example 1.

FIG. 2 is a picture that illustrates a printing result according to Example 1.

FIG. 3 is a picture that illustrates a printing result according to Example 2.

FIGS. 4 to 7 are pictures that illustrate printing results according to Example 3.

FIG. 8 is a picture that illustrates a printing result according to Example 4.

FIG. 9 is a picture that illustrates a printing result according to Example 5.

FIG. 10 is a picture that illustrates a printing result according to Example 6.

FIG. 11 is a picture that illustrates a printing result according to Comparative Example 2.

### BEST MODE

Hereinafter, the present invention will be described in detail.

An exemplary embodiment of the present invention provides a printing composition comprising an ionic liquid.

The ionic liquid includes an ion or short-lived ion pairs, and means a material existing in a liquid state at room temperature. This is different from that a general solvent maintains a liquid state in a molecular state that is electrically neutral. In the exemplary embodiment of the present invention, it was found that it is possible to provide a printing composition for forming a pattern having a fine line width by adding the ionic liquid to the printing composition.

In the case where only the solvent is used in the printing composition, in general, an organic solvent used in printing promotes drying of the ink composition due to a volatilization characteristic and absorption into silicon, such that when the pattern of the fine line width is formed, a residual film may be easily generated or poor patterns according to printing non-uniformity may be easily formed. However, in the exemplary embodiment of the present invention, the above problems may be prevented by adding the ionic liquid to the printing composition.

In addition, since the ionic liquid may be formed of a combination of various cations and anions, there is an advantage in that it is easy to control the physical properties thereof. For example, a viscosity, hydrophilicity, hydrophobicity, and stability of the ionic liquid may be controlled according to a kind of cation or anion, and in the case where the solvent is used together, miscibility with the solvent may be controlled.

For example, in the case of 1-methyl-3-alkyl-imidazolium hexafluorophosphate and 1-methyl-3-alkyl-imidazolium bis(trifluoromethylsulfonyl imide), the solubility to water when the anion is hexafluorophosphate is much higher than the solubility to water when the anion is bis(trifluoromethylsulfonyl imide) in respect to the same cation, and in the case of the same anion, as the length of the alkyl chain in the cation 1-methyl-3-alkyl-imidazolium becomes long, the solubility to water is decreased. In addition, in the case where 1-methyl-3-alkyl-imidazolium bis(trifluoromethylsulfonyl imide) is mixed in an acryl resin, the alkyl group becomes long, such that movement at a high temperature is largely reduced. As described above, the desired physical properties may be controlled by selecting various pairs of cation and anion.

In the exemplary embodiment of the present invention, since a vapor pressure may be negligible, drying to the air hardly occurs, and it is preferable to use the ionic liquid that is not absorbed into the blanket material, such as silicon resin. For example, in the case where the silicon resin is used as the blanket material, it is preferable that the ionic liquid has the high polarity unlike nonpolarity of the silicon resin.

The ionic liquid defines a salt that exists in a liquid state at a melting point temperature of 100° C. or less so that the salt exists in a liquid state at room temperature, and in the exemplary embodiment of the present invention, it is preferable that the salt in the liquid state at room temperature is a room temperature ionic liquid.

It is preferable that a boiling point of the ionic liquid is 300° C. or more. In this case, the volatilization characteristic into the air is largely reduced. In general, if the boiling point is increased, the vapor pressure is decreased.

The absorptivity (SP) into the silicon resin of the ionic liquid used as a blanket material in a general printing method is preferably about 1 or less and more preferably 0.5 or less. The absorptivity (SP) into the silicon resin may be represented by the following Equation.

SP=(length after immersion−length before immersion)/(length before immersion)×100

As the cations included in the ionic liquid, there are the following Formulas, but the scope of the present invention is not limited thereto.

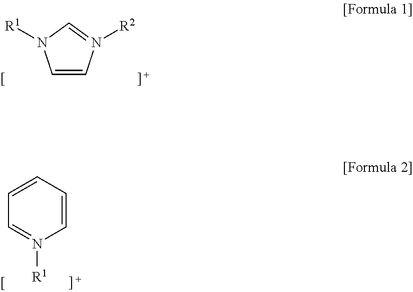

[NR¹R²R³R⁴]⁺ [Formula 3]

[PR¹R²R³R⁴]⁺ [Formula 4]

It is preferable that R¹ to R⁴ are independently a group including an ether bond, an alkyl group having 10 or less carbon atoms, or hydrogen.

As the anion included in the ionic liquid, for example, there are AlCl₄, Cl, Br, I, NO₃, SO₄, CF₃COO, CF₃SO₃, BF₄, PF₆, SbF₆ and [X(YOₘR_{f})ₙ] (R_{f} is a perfluoroalkyl group having 1 to 4 carbon atoms, Y is C or S, X is N or C, m is 1 in the case where Y is C and 2 in the case where Y is S, and n is 2 when X is nitrogen and 3 when X is carbon), but the scope of the present invention is not limited thereto.

It is preferable that a matter having excellent compatibility to other components added to the printing composition is used as the ionic liquid, and as preferable examples thereof, 1-butyl-3-methyl-imidazolium acetate, 1-ethyl-3-methyl-imidazolium acetate, 1-butyl-1-methyl-pyrrolidinium bis(trifluoromethylsulfonyl)imide, 1-butyl-3-methyl-imidazolium ethylsulfate, 1-butyl-3-methyl-imidazolium methanesulfonate, 1-butyl-3-methyl-imidazolium trifluoromethanesulfonate, 1-ethyl-3-methyl-imidazolium trifluoromethanesulfonate, 1-butyl-3-methyl imidazolium hexafluorophosphate, and 1-butyl-3-methyl imidazolium tetraborate may be used.

The physical properties of the solvents having excellent solubility in isopropanol among the ionic liquids are described in Table 1.

TABLE 1

| | | MP | Viscosity | Density |
|---|---|---|---|---|
| Ionic liquid | SP | (°) | (mm/s) | (g/ml) |
| 1-ethyl-3-methyl imidazolium | 0 | 11 | 34 | 1.24 |
| tetrafluoroborate | | | | |
| 1-butyl-1-methylpyrrolidinium | 0 | −50 | 71 | 1.4 |
| bis(trifluoromethyl sulfonyl)imide | | | | |
| 1-butyl-3-methyl imidazolium | 0 | 11 | 312 | 1.36 |
| hexafluorophosphate | | | | |
| 1-butyl-3-methyl imidazolium tetraborate | 0 | −71 | 233 | 1.21 |

It is preferable that the printing composition according to the exemplary embodiment of the present invention is added to the solvent in consideration of the vapor pressure showing the degree of volatilization into an air, the degree of swelling, the attachment property to the blanket or a printing target substrate, the degree of drying of the final pattern, and easiness of the printing process. As examples of the basic print solvent, alcohols, ethers, ketones, and hydrocarbons may be used. In detail, alcohols including methanol, ethanol, isopropanol, 1-methoxypropanol, butanol, ethylhexyl alcohol, and terpineol; glycols such as ethylene glycol, and glycerin; acetates such as ethyl acetate, butyl acetate, methoxypropylacetate, carbitol acetate, and ethylcarbitol acetate; ethers including methylcellosolve, butylcellosolve, diethyl ether, tetrahydrofuran, and dioxane; ketones including methyl ethyl ketone, acetone, dimethyl formamide, and 1-methyl-2-pyrrolidone; hydrocarbons including heptane, dodecane, paraffin oil, hydrocarbon series such as mineral spirits, benzene, toluene, and xylene; and a mixture solvent of two or more kinds may be used.

In the case where the printing composition according to the exemplary embodiment of the present invention further includes the solvent, it is preferable that the ionic liquid is 0.1 wt % to 10 wt % on the basis of the total printing composition. In this case, in the case where the content is less than 0.1 wt %, the printing of the fine line width is not improved, and in the case where the content is more than 10 wt %, it negatively affects the physical properties of the print material.

The printing composition according to the exemplary embodiment of the present invention may further include a functional material in order to provide a function required in a target print pattern. For example, the printing composition according to the exemplary embodiment of the present invention may further include at least one kind of material of a conductive material, an insulating material, and a semiconductor material. In this case, as the cation or the anion of the ionic liquid, it is preferable that a matter that does not cause a reaction with the functional material is selected.

As the conductive material, an organic metal salt, and metal particles may be used. For example, silver, aluminum, copper, neodymium, molybdenum, or an alloy thereof may be used. An organic binder for processability, glass frit, metal oxide as a blackening material, carbon black, carbon nanotube, a black pigment, and colored glass fit may be further added to the conductive material.

As the insulating material, a photocurable or heatcurable resin may be used. As the photocurable resin, a resin that is cured by irradiation of UV is used. For example, there are a resin composition that is formed of an acrylate compound having a radical reactive unsaturated bond, a resin composition that is formed of an acrylate compound having a radical reactive unsaturated bond and a mercapto compound, and a resin composition in which oligomer such as epoxy acrylate, urethane acrylate, polyester acrylate, and polyether acrylate is applied to a multifunctional acrylate monomer. As the thermosetting resin, there are a phenol resin, an unsaturated polyester resin, an epoxy resin, a polyurethane resin, a silicon resin, or a polyimide resin.

As the semiconductor material, for example, a single metal oxide and a compound having a Perovskite structure may be used as an oxide photosemiconductor. As the single metal oxide, there are titanium, tin, zinc, iron, tungsten, zirconium, hafnium, strontium, indium, cerium, yttrium, lanthanum, vanadium, niobium, or tandal oxide. As the compound having the Perovskite structure, there are strontium titanate, calcium titanate, sodium titanate, valium titanate, and potassium neobnate.

The printing composition including the ionic liquid according to the exemplary embodiment of the present invention may further include a solvent and a functional material.

In the printing composition including the ionic liquid, the solvent, and the functional material, on the basis of a total weight of the printing composition, 0.01 to 10 wt % of the ionic liquid, 5 to 80 wt % of the solvent, and 15 to 90 wt % of the functional material may be included.

In this case, in the case where the content of the ionic liquid is less than 0.1 wt %, the printing of the fine line width is not improved, and in the case where the content is more than 10 wt %, it negatively affects the physical properties of the print material. In addition, in the case where the content of the solvent is less than 5 wt %, the viscosity is too high, such that it is impossible to perform the printing process, and in the case where the content is more than 80 wt %, the viscosity is too low, such that it is impossible to perform the printing process and it is difficult to have a function as a print ink. In addition, in the case where the content of the functional material is less than 15 wt %, it is difficult to have a function as a print ink, and in the case where the content is more than 90 wt %, the print ink cannot be manufactured and the viscosity is too low, such that it is impossible to perform the printing process.

It is preferable that the printing composition according to the exemplary embodiment of the present invention has the viscosity of 3 cps to 30,000 cps.

Another exemplary embodiment of the present invention provides a printing method using the printing composition. The printing method used in the exemplary embodiment of the present invention is not particularly limited, and a gravure, gravure offset, reverse offset or inkjet method may be used, and in the printing method, a roll type or a plate type may be used.

For example, the reverse offset printing may be performed by coating a paste on a roll type blanket, closely contacting the paste and a cliché having unevenness to form a desired pattern on the blanket, and transferring the pattern formed on the blanket on a conductive film. In addition, the gravure offset printing may be performed by using the method in which after a paste is filled in an intaglio printing plate on which a pattern is formed, primary transferring is performed by using silicon rubber called a blanket, and secondary transferring is performed by closely contacting the blanket and a substrate on which a conductive film is formed. The intaglio printing plate may be manufactured by precisely etching the substrate. The intaglio printing plate may be manufactured by etching a metal plate, or may be manufactured through optical patterning using a polymer resin. The gravure printing may be performed by using the method in which after a paste is filled in a pattern while a blanket where the pattern is formed on a roll is wound, the paste is transferred on the substrate on which a conductive film is formed. In the exemplary embodiment of the present invention, the above methods may be used alone or in a combination.

Yet another exemplary embodiment of the present invention provides a pattern formed by using the printing composition.

The pattern formed according to the exemplary embodiment of the present invention has the particle property. Herein, the particle property means that functional materials configuring the pattern exist in a particle state as it is or in a state where the particles are necked to each other. This is compared to the case where the configuration materials of the pattern do not exist in the particle form like the case using a deposition method.

It is preferable that a ratio of a line width and a line height of the pattern formed according to the exemplary embodiment of the present invention (line height/line width) is 0.3 or less. The line width of the pattern may be 100 micrometers or less, preferably 0.1 to 30 micrometers, more preferably 0.5 to 20 micrometers, and more preferably 1 to 15 micrometers. The line height of the pattern may be 6 micrometers or less and preferably about 4 micrometers or less.

### MODE FOR INVENTION

The invention will be described in more detail in the following Examples. However, the following Examples are set forth to illustrate but are not to be construed to limit the present invention.

### Example 1

The printing composition (viscosity of 6000 cps) including the nanosilver paste (nanosilver 20 nm 70%, solvent 30%; the solvent is a mixture of alpha-terpineol and BCA (mixture of butyl carbitol acetate) and 5 wt % of 1-butyl-1-methyl pyrrolidinium bis(trifluoromethyl sulfonyl)imide on the total weight thereof was printed in the gravure offset manner at the relative humidity of 44%. In this case, the time from off to setting (hereinafter, referred to as a waiting time; Off is that all pastes on the cliché are transferred onto the silicon blanket, and Setting is that the paste on the blanket is transferred onto the substrate. Accordingly, the time after off is finished and directly before Setting starts is the waiting time was 0 sec. The printing result was shown in FIG. 2.

### Example 2

The same process as Example 1 was performed, except that the waiting time was 30 sec. The printing result was shown in FIG. 3. Even though the waiting time was 30 sec, it could be confirmed that the printing state was good.

### Example 3

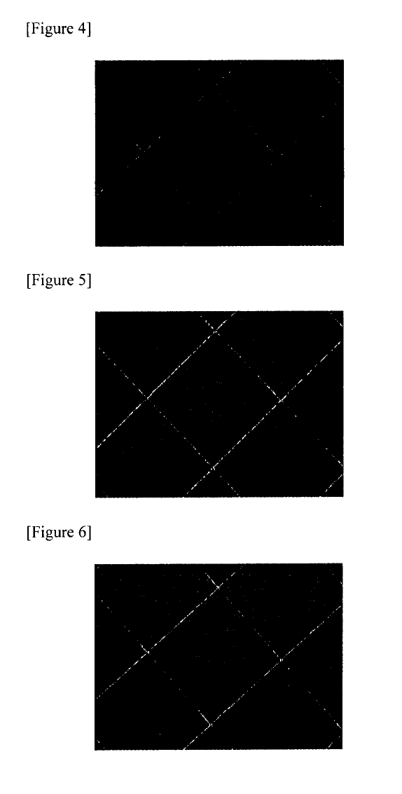

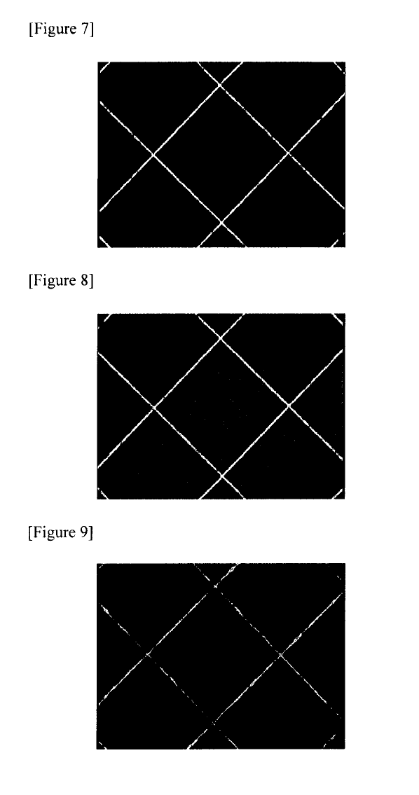

The printing was performed in the same manner as Example 1, except that the waiting time was fixed to 0 sec and the composition of the ionic liquid was changed to 0.1, 0.5, and 1.5%. The printing results were shown in FIGS. 4 to 7. It could be confirmed that the printing state was good in the range of the composition.

### Example 4

The same experiment as Example 1 was performed, except that 1-butyl-3-methyl-imidazolium tetraborate was used as the ionic liquid. The experiment result after the addition was shown in FIG. 8. It could be confirmed that the printing state became good after the ionic liquid was added thereto.

### Example 5

The same experiment as Example 1 was performed, except that 1-ethyl-3-methyl-imidazolium tetrafluoroborate was used as the ionic liquid. The experiment result after the addition was shown in FIG. 9. It could be confirmed that the printing state became good after the ionic liquid was added thereto.

### Example 6

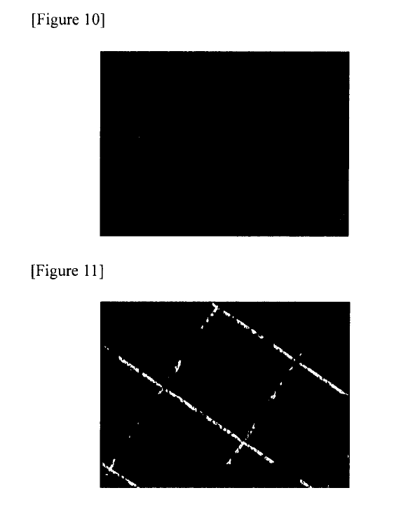

The same experiment as Example 1 was performed, except that 1-butyl-3-methyl-imidazolium hexafluorophosphate was used as the ionic liquid. The experiment result after the addition was shown in FIG. 10. It could be confirmed that the printing state became good after the ionic liquid was added thereto.

### Comparative Example 1

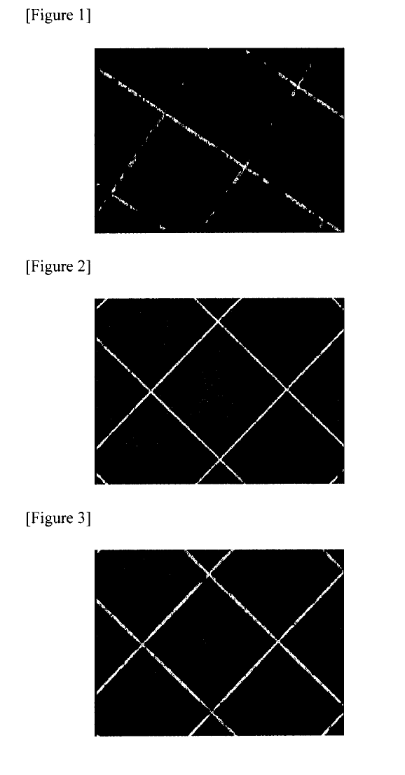

The same process as Example 1 was performed, except that 1-butyl-1-methyl pyrrolidinium bis(trifluoromethyl sulfonyl)imide that was the ionic liquid was not used. The printing result was shown in FIG. 1. Even though the waiting time was 0 sec, it could be confirmed that the poor printing occurred.

### Comparative Example 2

The same process as Example 1 was performed, except that propanol amine was used instead of 1-butyl-1-methylpyrrolidinium bis(trifluoromethyl sulfonyl)imide that was the ionic liquid. The printing result was shown in FIG. 11. Even though the waiting time was 0 sec, it could be confirmed that the poor printing occurred. SP of propanol amine was 8.

1. A printing composition comprising an ionic liquid.

2. The printing composition according to claim 1, further comprising a solvent.

3. The printing composition according to claim 2, wherein the ionic liquid is 0.1 to 10 wt % in a total printing composition.

4. The printing composition according to claim 2, wherein the solvent is alcohols including methanol, ethanol, isopropanol, 1-methoxypropanol, butanol, ethylhexyl alcohol and terpineol; glycols such as ethylene glycol and glycerin; acetates such as ethyl acetate, butyl acetate, methoxypropylacetate, carbitol acetate and ethylcarbitol acetate; ethers including methylcellosolve, butylcellosolve, diethyl ether, tetrahydrofuran and dioxane; ketones including methyl ethyl ketone, acetone, dimethyl formamide and 1-methyl-2-pyrrolidone; hydrocarbons including heptane, dodecane, paraffin oil, hydrocarbon series such as mineral spirits, benzene, toluene and xylene; and a mixture solvent of two or more kinds thereof.

5. The printing composition according to claim 1, further comprising a functional material.

6. The printing composition according to claim 5, wherein the functional material further includes at least one of a conductive material, an insulating material, and a semiconductor material.

7. The printing composition according to claim 1, further comprising a solvent and a functional material.

8. The printing composition according to claim 7, wherein on the basis of a total weight of the printing composition, 0.01 to 10 wt % of the ionic liquid, 5 to 80 wt % of the solvent, and 15 to 90 wt % of the functional material are included.

9. The printing composition according to claim 1, wherein a melting point of the ionic liquid is 100° C. or less.

10. The printing composition according to claim 1, wherein a boiling point of the ionic liquid is 300° C. or more.

11. The printing composition according to claim 1, wherein an absorptivity (SP) into a silicon resin of the ionic liquid represented by the following Equation is about 1 or less:
SP=(length after immersion−length before immersion)/(length before immersion)×100. [Equation]

12. The printing composition according to claim 1, wherein the ionic liquid includes one or more of cations represented by the following Formulas 1 to 4: 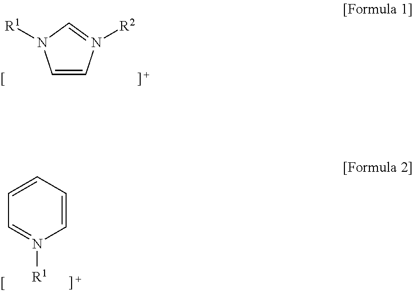
[NR¹R²R³R⁴]⁺ [Formula 3]
[PR¹R²R³R⁴]⁺ [Formula 4]
wherein R¹ to R⁴ are independently a group including an ether bond, an alkyl group having 10 or less carbon atoms, or hydrogen.

13. The printing composition according to claim 1, wherein the ionic liquid includes one or more anions selected from the group consisting of AlCl₄, Cl, Br, I, NO₃, SO₄, CF₃COO, CF₃SO₃, BF₄, PF₆, SbF₆, and [X(YOₘR_{f})ₙ] (R_{f} is a perfluoroalkyl group having 1 to 4 carbon atoms, Y is C or S, X is N or C, m is 1 in the case where Y is C, and is 2 in the case where Y is S, and n is 2 when X is nitrogen and is 3 when X is carbon).

14. The printing composition according to claim 1, wherein the ionic liquid includes one or more selected from the group consisting of 1-butyl-3-methyl-imidazolium acetate, 1-ethyl-3-methyl-imidazolium acetate, 1-butyl-1-methyl-pyrrolidinium bis(trifluoromethylsulfonyl)imide, 1-butyl-3-methyl-imidazolium ethylsulfate, 1-butyl-3-methyl-imidazolium methanesulfonate, 1-butyl-3-methyl-imidazolium trifluoromethanesulfonate, 1-ethyl-3-methyl-imidazolium trifluoromethanesulfonate, 1-butyl-3-methyl imidazolium hexafluorophosphate, and 1-butyl-3-methyl imidazolium tetraborate.

15. The printing composition according to claim 1, wherein a viscosity of the printing composition is 3 cps to 30,000 cps.

16. A printing method comprising:
printing the printing composition according to claim 1 on a substrate.

17. The printing method according to claim 16, wherein a gravure, gravure offset, reverse offset or inkjet method is used.

18. A pattern formed by using the printing composition according to claim 1.

19. The pattern according to claim 18, wherein the pattern has a particle property.

20. The pattern according to claim 18, wherein a ratio (line height/line width) of a line width and a line height of the pattern is 0.3 or less.

21. The pattern according to claim 18, wherein the line width of the pattern is 0.1 to 30 micrometers, and the line height is 6 micrometers or less.
